(19) United States
(12) Patent Application Publication
(10) Pub. No.: US 2015/0150766 A1
(43) Pub. Date: Jun. 4, 2015

(54) CATIONIC PARA-PHENYLENEDIAMINES, COMPOSITION COMPRISING AT LEAST SUCH COMPOUNDS, IMPLEMENTATION PROCESS THEREFOR AND USE THEREOF

(71) Applicant: L'OREAL, Paris (FR)

(72) Inventor: Aziz Fadli, Chelles (FR)

(21) Appl. No.: 14/365,705

(22) PCT Filed: Dec. 17, 2012
(86) PCT No.: PCT/EP2012/075827

§ 371 Date: Jun. 16, 2014

Related U.S. Application Data
(60) Provisional application No. 61/593,439, filed on Feb. 1, 2012.

(30) Foreign Application Priority Data
Dec. 16, 2011 (FR) 1161840

(51) Int. Cl. A61K 8/41; A61Q 5/10; A61K 8/49; C07C 217/84; C07D 295/135

(52) U.S. Cl. CPC A61K 8/416; C07C 217/84; C07D 295/135; A61K 8/4926; A61K 8/494; A61Q 5/10; A61K 2800/88; A61K 2800/43

(57) ABSTRACT

The present invention relates to particular cationic paraphenylenediamine compounds of formula (I) below and also to the use thereof for dyeing keratin fibres, in particular human keratin fibres such as the hair. The invention also relates to a dye composition comprising, in a suitable dyeing medium, one or more cationic para-phenylenediamines as defined previously. Finally, the invention relates to a dyeing device using the said composition.

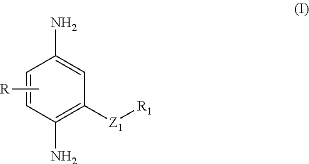

The present invention relates to particular cationic para-phenylenediamine compounds and also to the use thereof for dyeing keratin fibres, in particular human keratin fibres such as the hair.

The invention also relates to a composition for dyeing keratin fibres comprising such cationic para-phenylenediamine compounds in a suitable dyeing medium, and also to a dyeing process and a multi-compartment device using the said composition.

The present invention relates to the field of dyeing keratin fibres and more particularly to the field of hair dyeing.

It is known practice to dye keratin fibres, and in particular human hair, with dye compositions containing oxidation dye precursors, which are generally known as oxidation bases, such as ortho- or para-phenylenediamines, ortho- or para-aminophenols and heterocyclic compounds. These oxidation bases are colourless or weakly coloured compounds, which, when combined with oxidizing products, are able to produce coloured compounds by a process of oxidative condensation.

It is also known that the shades obtained with oxidation bases can be varied by combining them with couplers or coloration modifiers, the latter being selected more particularly from aromatic meta-diaminobenzenes, meta-aminophenols, meta-diphenols and certain heterocyclic compounds such as indole compounds.

The variety of molecules used as oxidation bases and couplers allows a wide range of colours to be obtained.

The “permanent” coloration obtained by means of these oxidation dyes must moreover satisfy a certain number of requirements. Thus, it must have no toxicological drawbacks, it must allow shades to be obtained in the desired strength, and it must show good fastness with respect to external agents such as light, bad weather, washing, permanent-waving, perspiration and rubbing.

The dyes are also required to cover white hairs, and to be extremely unselective, in other words to produce the smallest possible differences in coloration along a single stretch of keratin fibre, which in general has a sensitivity (in other words, damage) which differs between its tip and its root.

It is also known practice to use oxidation bases of the para-phenylenediamine type for dyeing keratin fibres, especially the hair. However, these oxidation bases generally have the drawback of giving colorations that are not sufficiently strong, chromatic or fast with respect to external agents and/or that are too selective.

There is thus a real need for oxidation bases that have improved dyeing properties, especially in terms of strength, chromaticity, colour buildup, selectivity and/or resistance with respect to external agents, and that are also capable of leading to a wide range of colours, while at the same time having improved harmlessness.

These aims are achieved with the present invention, a subject of which is especially cationic para-phenylenediamines of formula (I), optical isomers and geometrical isomers thereof, addition salts thereof and/or solvates thereof:

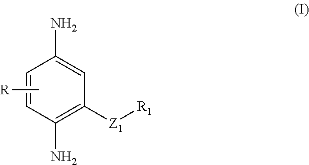

formula (I) in which:
R represents a hydrogen atom or a halogen atom; a C₁-C₄ alkyl radical; a carboxyl radical; a (C₁-C₄)alkoxycarbonyl radical;
Z₁ represents an oxygen atom or a group NR₆;
R₆ represents a hydrogen atom or a linear or branched C₁-C₄ alkyl radical;
R₁ represents:
   a linear or branched C₁-C₁₀ alkyl radical, substituted with a cationic radical, the said alkyl radical being optionally interrupted with one or more oxygen atoms and/or with one or more groups NR₆, optionally substituted with one or more radicals chosen from hydroxyl and C₁-C₄ alkoxy and hydroxyalkyl radicals;
   a saturated, unsaturated or aromatic, 5- to 8-membered non-cationic ring or heterocycle substituted with a cationic radical and optionally substituted with one or more radicals chosen from hydroxyl and C₁-C₄ alkoxy or hydroxyalkyl radicals;
when Z₁ represents NR₆ then
   R₁ and R₆ may form, together with the nitrogen atom to which they are attached, a saturated, unsaturated, aromatic or non-aromatic, 5- to 8-membered mono- or polycationic, preferably monocationic, heterocycle, optionally substituted with one or more radicals chosen from C₁-C₄ alkyl, hydroxyl, C₁-C₄ alkoxyamino, (C₁-C₄)alkylamino, (C₁-C₄)dialkylamino, thio SH, (C₁-C₄)alkylthio, carboxyl, C(O)OH or C(O)O—, M+ with M+, which may be present or absent depending on the cationic charge number of the compound (I), representing an alkali metal, alkaline-earth metal or ammonium; (C₁-C₄)alkylcarbonyl; sulfonyl —S(O)ₙ—R, —S(O)ₚ—O—R, —O—S(O)ₚ—R with R representing a hydrogen atom or a C₁-C₄ (hydroxy)alkyl group, n=0, 1 or 2, p=1 or 2, amido —C(O)—NRR′ or —N(R)—C(O)—R′, —N(R)—C(O)—NRR′ with R and R′, which may be identical or different, representing a hydrogen atom or a C₁-C₄ (hydroxy)alkyl group, C₁-C₄ hydroxyalkyls, this heterocycle possibly containing one or more heteroatoms chosen from N and O, preferably N,
   R₁ and R₆ may form, together with the nitrogen atom to which they are attached, a saturated or unsaturated, 5- to 8-membered noncationic heterocycle optionally substituted with one or more radicals chosen from C₁-C₁₀ alkyl; hydroxyl OH; C₁-C₄ alkoxy; amino; (C₁-C₄)alkylamino; (C₁-C₄)dialkylamino; thio SH, (C₁-C₄)alkylthio, carboxyl, C(O)OH or C(O)O—, M⁺ with M⁺, which may be present or absent depending on the cationic charge number of the compound (I), representing an alkali metal, alkaline-earth metal or ammonium; (C₁-C₄)alkylcarbonyl; sulfonyl —S(O)ₙ—R, —S(O)ₚ—O—R, —O—S(O)ₚ—R with R representing a hydrogen atom or a C₁-C₄ (hydroxy)alkyl group, n=0, 1 or 2, p=1 or 2, amido —C(O)—NRR′ or —N(R)—C(O)—R′, —N(R)—C(O)—NRR′ with R and R′, which may be identical or different, representing a hydrogen atom or a C₁-C₄ (hydroxy)alkyl group, C₁-C₄ hydroxyalkyls,

The invention also relates to the use of one or more cationic para-phenylenediamine compounds of formula (I) as defined previously, in the presence of one or more oxidizing agents, for dyeing keratin fibres, in particular human keratin fibres such as the hair.

The present invention also relates to a composition for dyeing keratin fibres, in particular human keratin fibres such as the hair, comprising, in a suitable dyeing medium, one or more cationic para-phenylenediamine compounds of formula (I) as defined previously.

In particular, the invention also relates to the use of the said composition for dyeing keratin fibres, in particular human keratin fibres such as the hair.

The invention also relates to a process for dyeing keratin fibres, in particular human keratin fibres such as the hair, in which the said dye composition according to the invention is applied to the said fibres in the presence of one or more oxidizing agents for a time that is sufficient to obtain the desired coloration, after which the resulting fibres are rinsed, optionally washed with shampoo, rinsed again and dried or left to dry.

Another subject of the present invention concerns a multi-compartment device or dyeing kit comprising a first compartment containing a dye composition as described above and a second compartment containing one or more oxidizing agents.

The multi-compartment device is thus suitable for performing the dyeing process according to the invention.

The cationic para-phenylenediamine compounds according to the invention thus make it possible to obtain improved dyeing of keratin fibres, especially in terms of strength or chromaticity, and/or of selectivity and/or of resistance to external agents such as shampoos, sweat, light and permanent reshaping.

The cationic para-phenylenediamine compounds according to the invention make it possible to achieve a wide range of colours, while at the same time improving the harmlessness of the oxidation bases.

Furthermore, cationic para-phenylenediamine compounds according to the invention show good solubility and allow satisfactory colour buildup.

For the purposes of the present invention, the term “buildup” of the colour of keratin fibres means the variation in coloration between locks of undyed grey hair and locks of dyed hair.

Other features, aspects, subjects and benefits of the present invention will emerge even more clearly on reading the description and the examples that follow.

I. Cationic Para-Phenylenediamine Compounds

The para-phenylenediamine compounds of formula (I) as defined previously comprise a cationic charge by virtue of the presence of a cationic radical in their structure.

In the context of the invention, the expression “cationic radical present in the compound of formula (I)” means any linear or branched or heterocyclic radical comprising a quaternary ammonium, this quaternary ammonium being of the type —N⁺RₐR_{b}R_{c}, Rₐ, R_{b} and R_{c}, which may be identical, representing a C₁-C₆ alkyl radical which may be substituted with a hydroxyl or Rₐ and R_{b} may together form a 5- to 8-membered heterocycle, the radical R_{c} then being a C₁-C₆ alkyl radical which may be substituted with a hydroxyl radical.

Preferably, Rₐ, R_{b} and R_{c}, which are identical, represent a C₁-C₂ alkyl radical, in particular methyl, which may be substituted with a hydroxyl radical.

Preferably, when Rₐ and R_{b} together form a 5- to 8-membered heterocycle, the radical R_{c} then represents a C₁-C₂ alkyl radical which may be substituted with a hydroxyl radical.

Thus, the para-phenylenediamines of formula (I) according to the invention bear a permanent cationic charge that is independent of the pH of the medium in which the compounds are formulated.

Examples of cationic radicals that may be mentioned include trimethylammonium, triethylammonium and tri-β-hydroxyethylammonium radicals.

The term “cationic heterocycle” means a 5- to 8-membered heterocycle in which at least one of the chain members is a quaternary ammonium as defined previously.

In particular, the cationic heterocycle is a 5- to 8-membered heterocycle in which at least one of the chain members is a quaternary ammonium as defined previously.

Examples of cationic heterocycles that may be mentioned include piperaziniums, pyrrolidiniums, morpholiniums, pyrazoliums, for example pyrrolidine, piperidine, morpholine and piperazine rings.

These cationic heterocycles are optionally substituted with one or more identical or different radicals chosen from (C₁-C₄)alkyl and (C₁-C₄)hydroxyalkyl radicals.

Preferably, cationic radicals are chosen from trimethylammonium, triethylammonium, and cationic heterocycles such as piperaziniums, pyrrolidiniums and morpholiniums.

More preferentially, the cationic radicals are chosen from trimethylammonium radicals and cationic heterocycles, especially piperazinium, pyrrolidinium, piperidinium and morpholinium.

Even more preferentially, the cationic radicals are chosen from trimethylammonium, piperazinium, in particular methylpiperazinium and dimethylpiperazinium.

The term “saturated, unsaturated or aromatic 5- to 8-membered non-cationic heterocycle” means a 5- to 8-membered ring in which at least one of the ring members is a heteroatom chosen from O, N and S, for instance imidazole or pyrimidine.

In particular, the cationic heterocycle is a 5- to 8-membered heterocycle in which at least one of the chain members is a quaternary ammonium as defined previously.

According to one preferred embodiment, in formula (I), are such that, taken together or separately:

R represents a hydrogen atom or a C₁-C₄ alkyl radical, and more preferentially R represents a hydrogen atom,

Z₁ represents an oxygen atom or a group NR₆ with R₆ representing a hydrogen atom or a C₁-C₂ alkyl radical, and more preferentially, R₆ represents a hydrogen atom or a methyl radical,

R₁ represents a linear or branched C₁-C₈ alkyl radical, substituted with a cationic radical, optionally interrupted with one or more oxygen atoms and/or with one or more groups NR₆, optionally substituted with a hydroxyl radical, the said cationic radical being optionally substituted with one or more radicals chosen from C₁-C₄ alkyl and C₁-C₄ alkoxy radicals,

or

R₁ represents a saturated, unsaturated or aromatic, 5- to 8-membered non-cationic ring substituted with a cationic radical and optionally substituted with one or more radicals chosen from hydroxyl and C₁-C₄ alkoxy or hydroxyalkyl radicals,

and when Z₁ represents NR₆ then
R₁ and R₆ may form, together with the nitrogen atom to which they are attached, a saturated or unsaturated cationic 5- to 8-membered heterocycle optionally substituted with one or more radicals chosen from C₁-C₁₀ alkyl, hydroxyl, C₁-C₄ alkoxy, amino, (C₁-C₄)alkylamino, di(C₁-C₄)alkylamino, thio, (C₁-C₄)alkylcarbonyl, amido and C₁-C₄ hydroxyalkyl radicals, it being possible for this heterocycle to contain one or more heteroatoms chosen from N or O, preferably N,
or
R₁ and R₆ may form, together with the nitrogen atom to which they are attached, a saturated or unsaturated non-cationic 5- to 8-membered heterocycle substituted with a cationic radical and optionally substituted with one or more radicals chosen from C₁-C₁₀ alkyl, hydroxyl, C₁-C₄ alkoxy, amino, (C₁-C₄)alkylamino, di(C₁-C₄)alkylamino, (C₁-C₄)alkylcarbonyl, amido and C₁-C₄ hydroxyalkyl radicals.

Preferably, R₁ represents a linear or branched C₁-C₈ alkyl radical, substituted with a cationic radical, optionally interrupted with one or more oxygen atoms and/or with one or more groups NR₆.

Preferably, when Z₁ represents a group NR₆, then R₁ and R₆ may form, together with the nitrogen atom to which they are attached, a saturated or unsaturated, 5- to 8-membered cationic heterocycle, optionally substituted with one or more radicals chosen from C₁-C₄ alkyl and C₁-C₄ hydroxyalkyl radicals, this heterocycle possibly containing a nitrogen atom.

According to a first preferred variant of the invention, R denotes a hydrogen atom, Z₁ represents an oxygen atom or a group NH or NCH₃, R₁ represents a saturated linear C₂-C₈ alkyl radical, optionally interrupted with a heteroatom such as oxygen or a group NH, R₁ being substituted with a cationic radical chosen from trimethylammonium radicals and piperazinium, pyrrolidinium, piperidinium or morpholinium cationic heterocycles.

According to the second preferred variant of the invention, R denotes a hydrogen atom, Z₁ is a group NR₆ and R₁ and R₆ form, together with the nitrogen atom to which they are attached, a piperazinium, pyrrolidinium, piperidinium or morpholinium radical substituted with one or more identical or different radicals chosen from C₁-C₄ hydroxyalkyl and C₁-C₄ alkyl, preferentially a piperazinium radical substituted with one or more identical or different radicals chosen from C₁-C₄ hydroxyalkyl and C₁-C₄ alkyl.

According to a third preferred variant of the invention, R denotes a hydrogen atom, Z₁ is a group NR₆ and R₁ and R₆ form, together with the nitrogen atom to which they are attached, a saturated or unsaturated, 5- to 8-membered non-cationic heterocycle, substituted with a cationic radical preferably chosen from trimethylammonium, piperazinium, piperidinium, pyrrolidinium and morpholinium radicals. In accordance with this variant and even more preferentially, the non-cationic heterocycle is chosen from pyrrolidine, piperidine and morpholine, this ring being substituted with a cationic radical chosen from trimethylammonium and radicals. In accordance with this variant and even more preferentially, the non-cationic heterocycle is chosen from pyrrolidine, piperidine and morpholine, this ring being substituted with a cationic radical chosen from trimethylammonium radical.

According to one embodiment, Z represents an oxygen atom.

According to another embodiment, Z represents a group NR₆, in which R₁ and R₆ form, together with the nitrogen atom to which they are attached, a saturated or unsaturated, 5- to 8-membered cationic heterocycle, optionally substituted with one or more radicals chosen from C₁-C₄ alkyl and C₁-C₄ hydroxyalkyl radicals, this heterocycle containing a nitrogen atom other than that belonging to the group NR₆.

In the context of the invention, a derivative of formula (I) is understood to encompass all mesomeric or isomeric forms.

The electrical neutrality of the compounds of formula (I) is ensured by an organic or mineral, cosmetically acceptable anion or mixture of anions, noted An⁻.

An⁻ denotes, for example, a halide such as chloride, bromide, fluoride or iodide; a hydroxide; a sulfate; a hydrogen sulfate; an alkyl sulfate in which the linear or branched alkyl part is of C₁-C₆, such as the methyl sulfate or ethyl sulfate ion; a carbonate; a hydrogen carbonate; a salt of a carboxylic acid, such as formate, acetate, citrate, tartrate or oxalate; an alkylsulfonate for which the linear or branched alkyl part is of C₁-C₆, such as the methylsulfonate ion; an arylsulfonate for which the aryl part, preferably phenyl, is optionally substituted with one or more C₁-C₄ alkyl radicals, for instance 4-tolylsulfonate; an alkylsulfonate such as mesylate.

Preferably, An⁻ is a halide, especially chloride.

The compounds of general formula (I) may be in free form or in the form of salts, such as addition salts with an inorganic acid preferably chosen from hydrochloric acid, hydrobromic acid, sulfuric acid and phosphoric acid or with an organic acid such as, for example, citric acid, succinic acid, tartaric acid, lactic acid, 4-toluylsulfonic acid, benzenesulfonic acid, acetic acid, para-toluenesulfonic acid, formic acid and methanesulfonic acid.

For the purposes of the present invention, the cationic charge originating from the acid salt of compound (I) is not considered as a cationic radical borne by Z₁.

The compounds of general formula (I) may also be in the form of solvates, for example in the form of a hydrate or of a solvate of a linear or branched C₁-C₆ alcohol such as ethanol or isopropanol.

Preferably, the cationic para-phenylenediamines of formula (I) according to the invention are chosen from the following compounds and mixtures thereof, and also the geometrical or optical isomer forms thereof, the organic or mineral acid salts thereof or the solvates thereof such as hydrates:

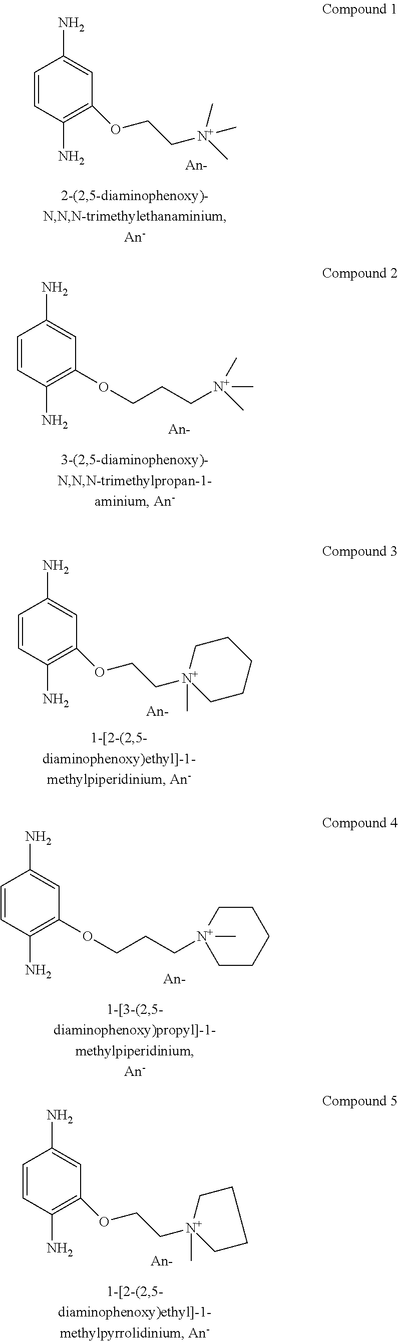

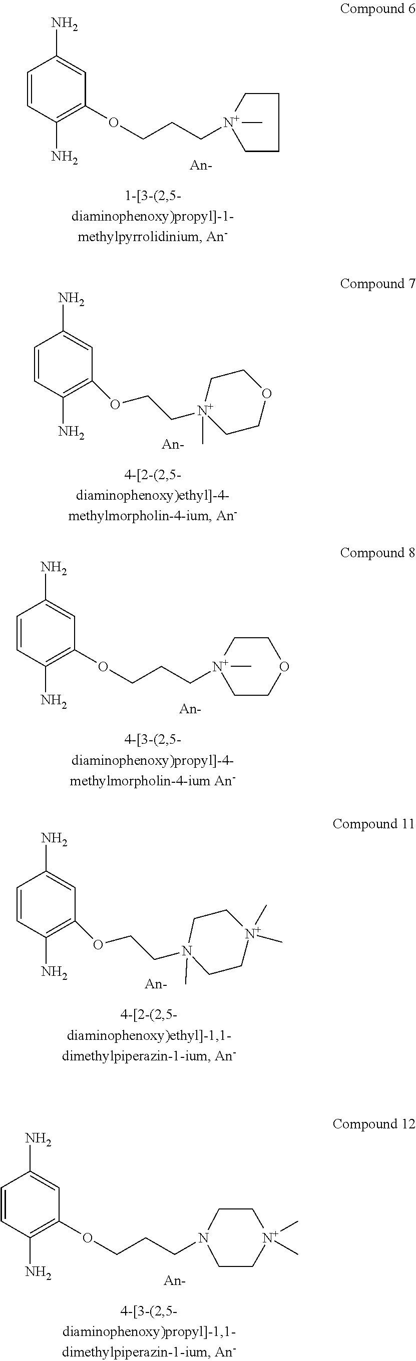

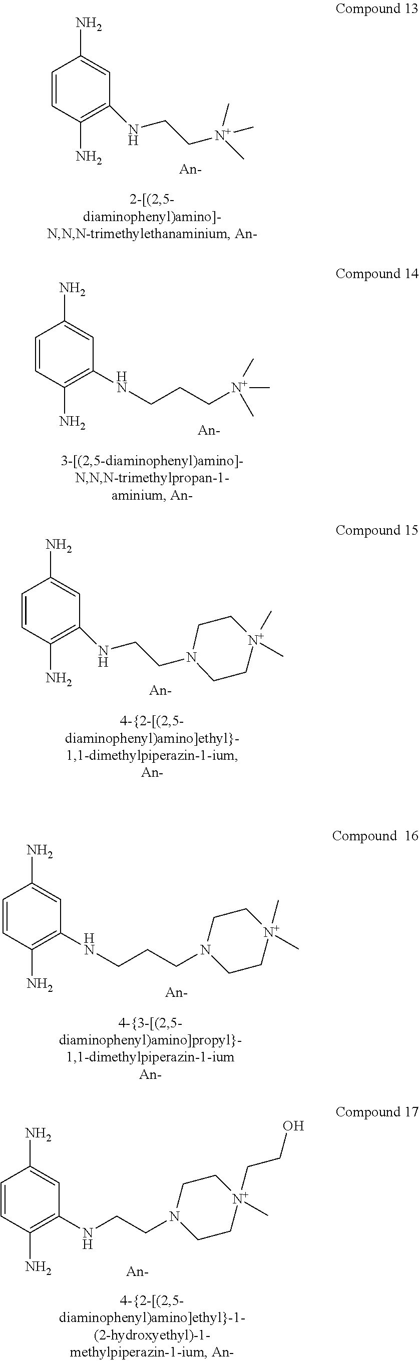 [SMILES: C[N+](C)(C)CCNc1cc(N)ccc1N ; C[N+](C)(C)CCCNc1cc(N)ccc1N ; C[N+]1(C)CCN(CCNc2cc(N)ccc2N)CC1 ; C[N+]1(C)CCN(CCCNc2cc(N)ccc2N)CC1 ; C[N+]1(CCO)CCN(CCNc2cc(N)ccc2N)CC1]

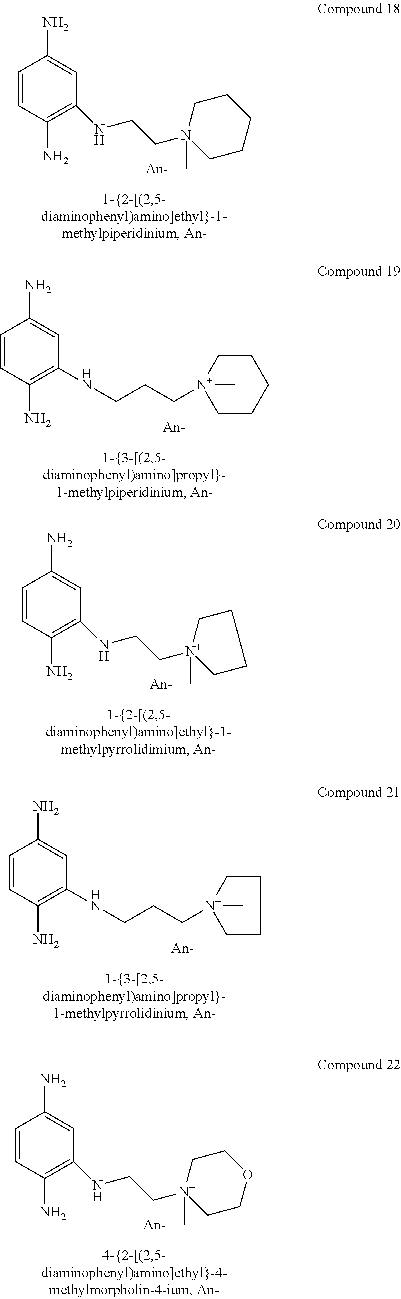

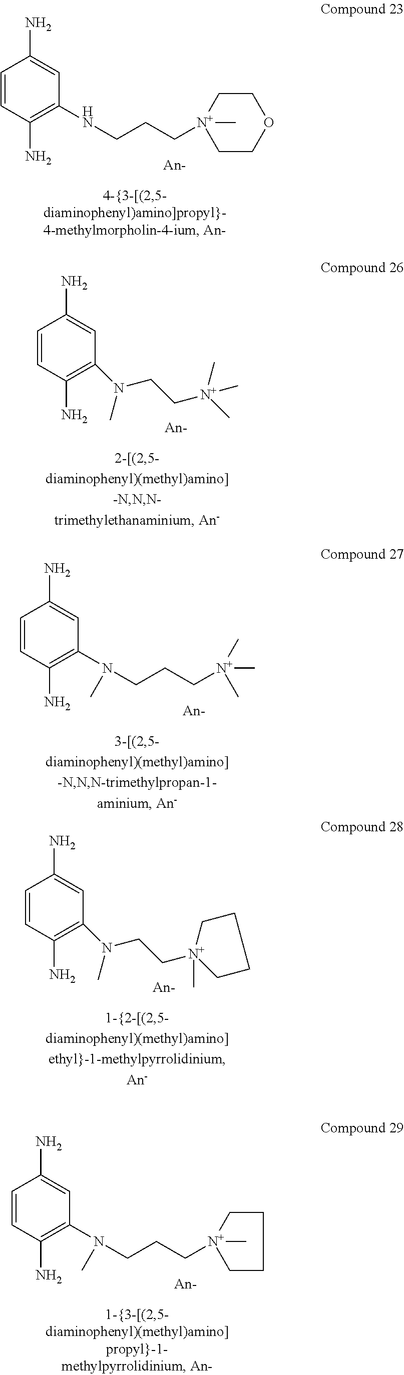

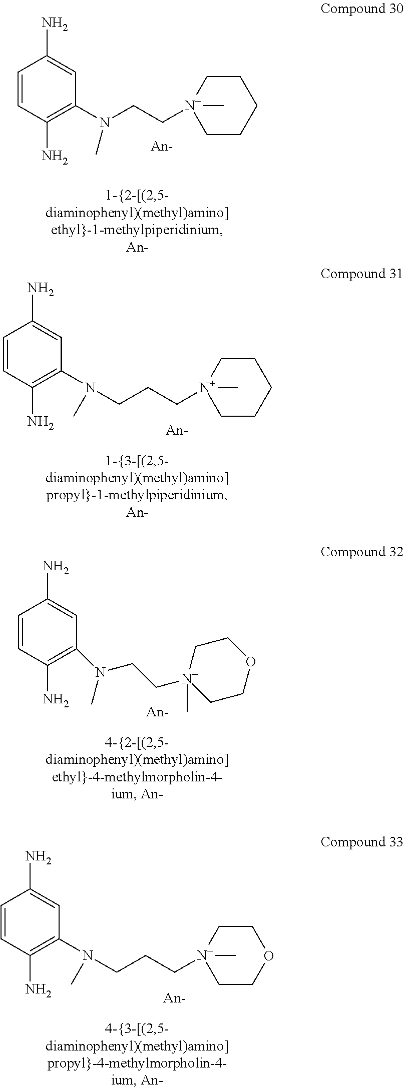

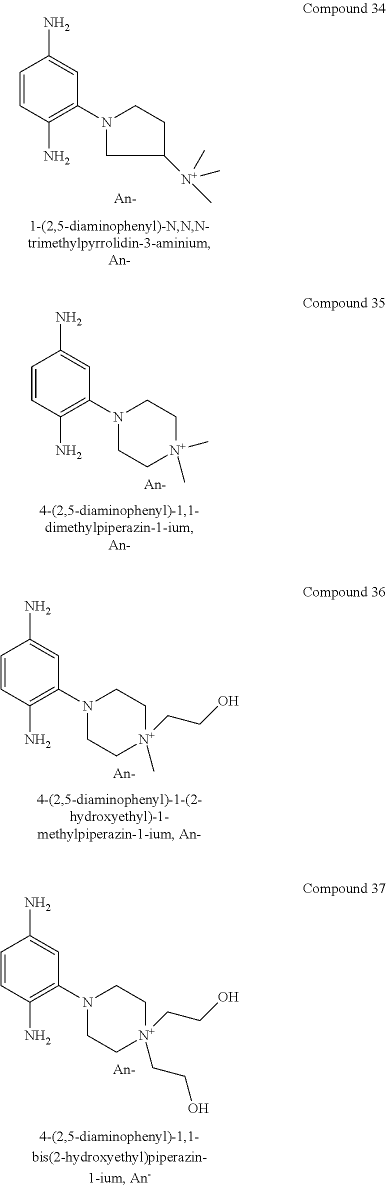 [SMILES: C[N+](C)(C)C1CCN(c2cc(N)ccc2N)C1 ; C[N+]1(C)CCN(c2cc(N)ccc2N)CC1 ; C[N+]1(CCO)CCN(c2cc(N)ccc2N)CC1 ; Nc1ccc(N)c(N2CC[N+](CCO)(CCO)CC2)c1]

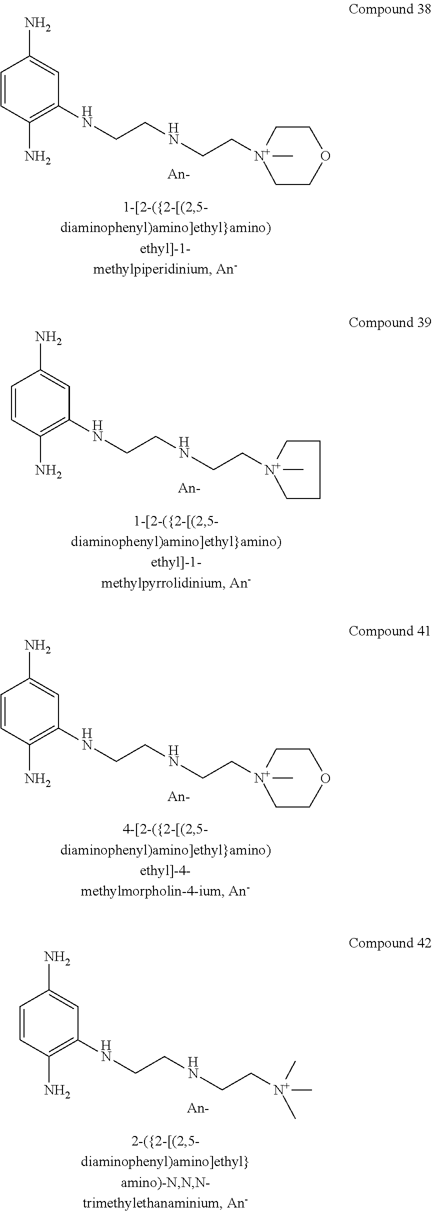

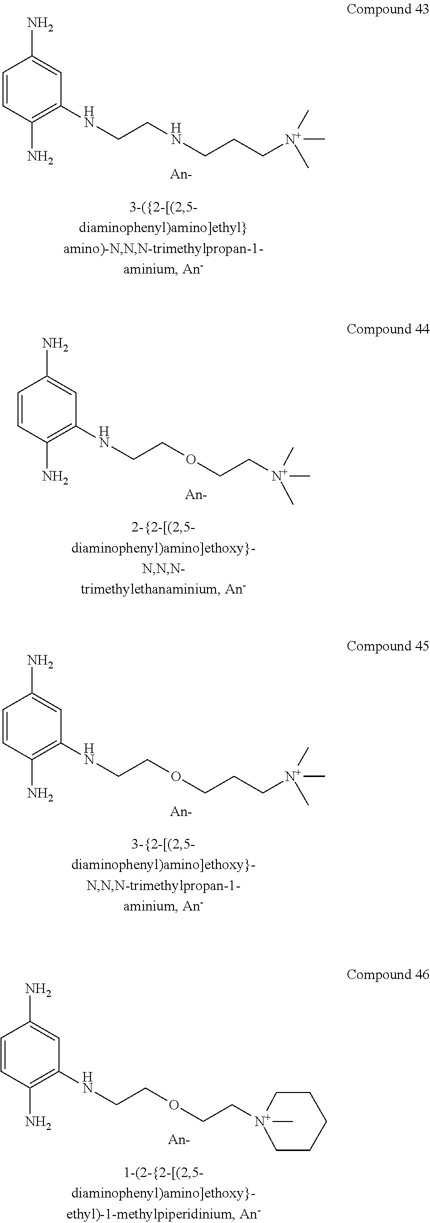

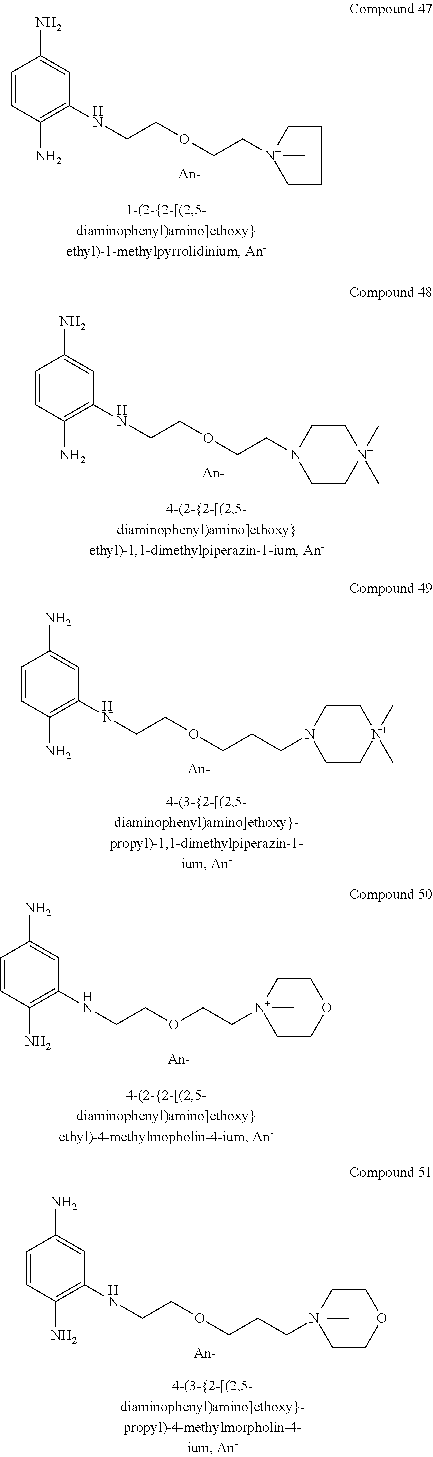

An⁻ having the same meaning as previously.

Preferably, the cationic para-phenylenediamines according to the invention are chosen from compounds 1, 3, 11 and 35, and also mixtures thereof.

In accordance with one embodiment, the cationic para-phenylenediamines in accordance with the present invention may be obtained according to the following synthetic scheme:
X=nucléofuge tel que halogène ou SO₂R

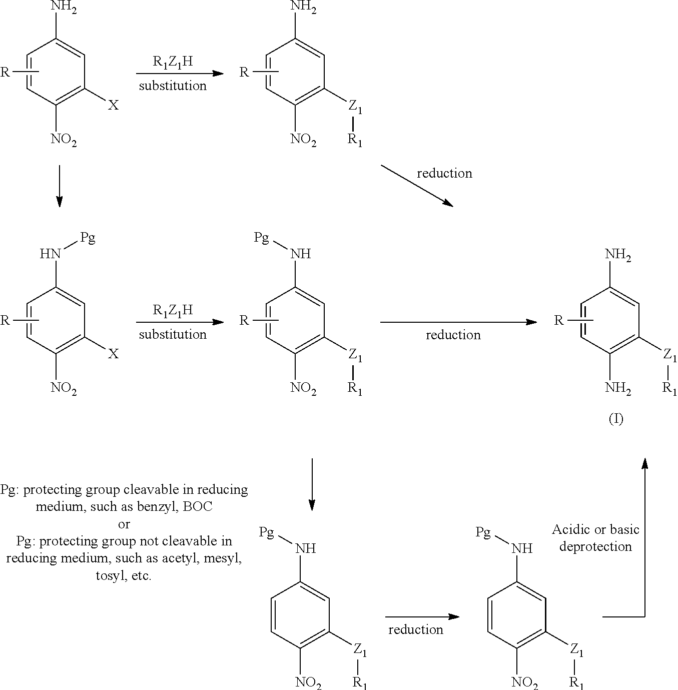

According to one embodiment, when R₁ represents a C₁-C₁₀ alkyl radical substituted with a cationic radical, the said alkyl radical being interrupted with one or more oxygen atoms and/or with one or more groups NR₆, then the synthetic process used may be the following:

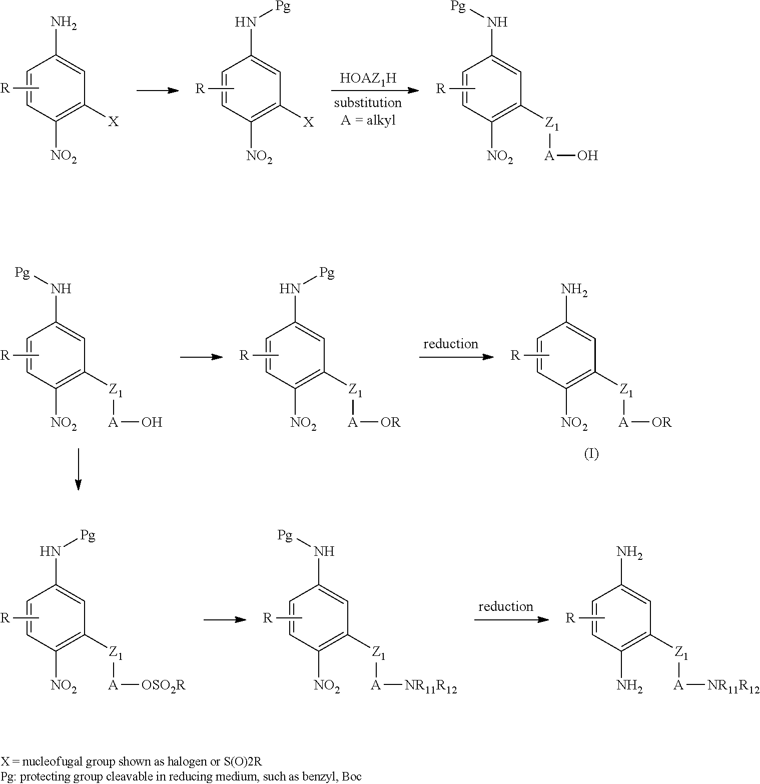

The substitution reaction is performed in a dipolar solvent such as acetonitrile, tetrahydrofuran (THF) or in dimethylformamide (DMF) or N-methylpyrrolidone (NMP), or in an alcohol such as ethanol, in the presence of a base such as triethylamine, ethyldiisopropylamine, sodium hydroxide or potassium hydroxide, for example, and one or more equivalents of HOAZ₁H for 1 to 24 hours at a temperature from 20° C. to the reflux temperature of the solvent.

The hydroxyl function thus introduced is then substituted with a halide (for example mesyl or tosyl halide) in a solvent such as acetonitrile or THF or in an alcohol such as ethanol, for example, in the presence of a base such as triethylamine, ethyldiisopropylamine, sodium hydroxide or potassium hydroxide, for example, for 1 to 24 hours at a temperature from 20° C. to the reflux temperature of the solvent.

The substitution of the leaving group introduced in the preceding step is performed either by reaction with an aromatic tertiary amine such as methylimidazole to lead directly to the cationic compounds, or by reaction with a particular primary or secondary amine, for instance N,N-dimethylethylenediamine or 2-piperidin-1-ylethanamine to lead to the compounds that are alkylated with at least one equivalent of alkyl halide or of methyl sulfate in a solvent such as THF or acetonitrile or dioxane or ethyl acetate for 15 minutes to 24 hours at a temperature ranging from 15° C. to the reflux temperature of the solvent, to give the cationic nitro compounds.

The reduction of the nitro group of these compounds is performed under standard conditions, for example by performing a hydrogenation reaction under heterogeneous catalysis in the presence of Pd/C, Pd(II)/C, Ni/Ra, etc., or alternatively by performing a reduction reaction with a metal, for example with zinc, iron, tin, etc. (see Advanced Organic Chemistry, 3rd Edition, J. March, 1985, Wiley Interscience and Reduction in Organic Chemistry, M. Hudlicky, 1983, Ellis Horwood Series Chemical Science).

The invention also relates to the use of one or more cationic para-phenylenediamine compounds of formula (I) as defined previously, in the presence of one or more oxidizing agents, for dyeing keratin fibres, in particular human keratin fibres such as the hair.

II. Dye Composition

The present invention also relates to a composition for dyeing keratin fibres, in particular human keratin fibres such as the hair, comprising, in a suitable dyeing medium, one or more cationic para-phenylenediamine compounds of formula (I) as defined above.

Preferably, the dye composition comprises one or more cationic para-phenylenediamine compounds of formula (I) chosen from compounds 1, 3, 11 and 35, and mixtures thereof.

The cationic para-phenylenediamines as defined previously may be present in the composition according to the invention in a content ranging from 0.1% to 20% by weight and preferably in a content ranging from 0.1% to 5% by weight relative to the total weight of the dye composition.

The dye composition according to the invention may contain and preferably contains one or more couplers that are conventionally used for dyeing keratin fibres. Among these couplers, mention may be made especially of meta-phenylenediamines, meta-aminophenols, meta-diphenols, naphthalene couplers and heterocyclic couplers, and the addition salts thereof.

Examples of couplers that may be mentioned include 2-methyl-5-aminophenol, 5-N-(β-hydroxyethyl)amino-2-methylphenol, 6-chloro-2-methyl-5-aminophenol, 3-aminophenol, 2,4-dichloro-3-aminophenol, 5-amino-4-chloro-o-cresol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 1,5-dihydroxynaphthalene, 2,7-naphthalenediol, 1-acetoxy-2-methylnaphthalene, 6-hydroxyindole, 4-hydroxyindole, 4-hydroxy-N-methylindole, 2-amino-3-hydroxypyridine, 6-hydroxybenzomorpholine 3,5-diamino-2,6-dimethoxypyridine, 2,6-dihydroxy-3-4-dimethyl pyridine, 3-amino-2-methylamino-6-methoxypyridine, 1-N-(β-hydroxyethyl)amino-3,4-methylenedioxybenzene, 2,6-bis(β-hydroxyethylamino)toluene and 3-methyl-1-phenyl-5-pyrazolone, and the addition salts thereof with an acid.

In the dye composition of the present invention, the coupler(s), if they are present, generally represent an amount of between 0.001% and 10% by weight approximately relative to the total weight of the composition, and preferably between 0.005% and 6% by weight relative to the total weight of the composition.

The dye composition of the invention may optionally comprise one or more additional oxidation bases conventionally used for the dyeing of keratin fibres, other than the compounds of formula (I).

By way of example, these additional oxidation bases are chosen from para-phenylenediamines other than the bases of formula (I), bis(phenyl)alkylenediamines, para-aminophenols, bis-para-aminophenols, ortho-aminophenols and heterocyclic bases, and the addition salts thereof.

Among the para-phenylenediamines, examples that may be mentioned include para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines, examples that may be mentioned include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols, examples that may be mentioned include para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 1-hydroxy-4-methylaminobenzene and 2,2′-methylenebis(4-aminophenol), and the addition salts thereof with an acid.

Among the ortho-aminophenols, examples that may be mentioned include 2-aminophenol, 2-amino-5-methylphenol, 2-amino-6-methylphenol and 5-acetamido-2-aminophenol, and the addition salts thereof with an acid.

Among the heterocyclic bases, examples that may be mentioned include pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof with an acid.

Among the pyrimidine derivatives, mention may be made of the compounds described, for example, in patents DE 2 359 399; JP 88-169 571; JP 05-63124; EP 0 770 375 or patent application WO 96/15765, for instance 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048, and among which mention may be made of pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,-N7,N7-tetramethylpyrazolo[1,5-a]pyrimidine-3,7-diamine and 3-amino-5-methyl-7-imidazolylpropylaminopyrazolo[1,5-a]pyrimidine, and the addition salts thereof with an acid, and the tautomeric forms thereof, when a tautomeric equilibrium exists.

Among the pyrazole derivatives that may be mentioned are the compounds described in the patents DE 3843892, DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof with an acid.

In general, the addition salts of the additional oxidation bases and of the couplers that can be used in the context of the invention are especially chosen from addition salts with an acid, such as hydrochlorides, hydrobromides, sulfates, citrates, succinates, tartrates, lactates, tosylates, benzenesulfonates, phosphates and acetates, and the addition salts with a base such as sodium hydroxide, potassium hydroxide, aqueous ammonia, amines or alkanolamines.

The dye composition in accordance with the invention may also contain one or more direct dyes that may in particular be chosen from nitrobenzene dyes, azo direct dyes and methine direct dyes. These direct dyes may be of nonionic, anionic or cationic nature.

The medium that is suitable for dyeing, also known as the dye support, generally comprises water or a mixture of water and of one or more organic solvents, for example C₁-C₄ lower alkanols such as ethanol and isopropanol, polyols, for instance propylene glycol, dipropylene glycol or glycerol, and polyols, for instance dipropylene glycol monomethyl ether.

The solvent(s) are generally present in proportions that may be between 1% and 40% by weight approximately and more preferably between 3% and 30% by weight approximately relative to the total weight of the dye composition.

The dye composition in accordance with the invention may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, and in particular anionic, cationic, nonionic and amphoteric polymeric associative thickeners, antioxidants, penetrants, sequestrants, fragrances, buffers, dispersants, conditioning agents, for instance volatile or non-volatile, modified or unmodified silicones, film-forming agents, ceramides, preserving agents and opacifiers.

The above adjuvants are generally present in an amount, for each of them, of between 0.01% and 20% by weight relative to the weight of the composition.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between 3 and 12 approximately and preferably between 5 and 11 approximately. It may be adjusted to the desired value by means of acidifying or basifying agents customarily used in the dyeing of keratin fibres, or alternatively using standard buffer systems.

Among the acidifying agents, mention made be made, by way of example, of inorganic or organic acids, for instance hydrochloric acid, orthophosphoric acid, sulfuric acid, carboxylic acids, for instance acetic acid, tartaric acid, citric acid and lactic acid, and sulfonic acids.

Among the basifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds of formula (II) below:

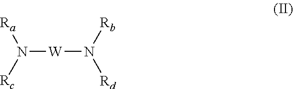

in which W is a propylene residue optionally substituted with a hydroxyl group or a C₁-C₄ alkyl radical; and Rₐ, R_{b}, R_{c} and R_{d}, which may be identical or different, represent a hydrogen atom or a C₁-C₄ alkyl or C₁-C₄ hydroxyalkyl radical.

The composition according to the invention may comprise one or more oxidizing agents.

The oxidizing agents are those conventionally used for the oxidation dyeing of keratin fibres, for example hydrogen peroxide, urea peroxide, alkali metal bromates, persalts such as perborates and persulfates, peracids and oxidase enzymes, among which mention may be made of peroxidases, 2-electron oxidoreductases such as uricases, and 4-electron oxygenases, for instance laccases. Hydrogen peroxide is particularly preferred.

The dye composition with or without oxidizing agent according to the invention may be in various forms, such as in the form of liquids, creams or gels, or in any other form that is suitable for dyeing keratin fibres, and especially human hair.

It may result from the mixing, at the time of use, of several compositions.

In particular, it results from the mixing of at least two compositions, one comprising one or more oxidation bases chosen from the compounds of formula (I) or addition salts thereof with an acid, optionally one or more additional oxidation bases other than the compounds of formula (I) or salts thereof, and optionally one or more couplers, and the second composition comprising one or more oxidizing agents as described previously.

The present invention also relates to a process for dyeing keratin fibres, in particular human keratin fibres such as the hair, in which the said dye composition according to the invention is applied to the said fibres in the presence of one or more oxidizing agents for a time that is sufficient to obtain the desired coloration, after which the resulting fibres are rinsed, optionally washed with shampoo, rinsed again and dried or left to dry.

The colour may be revealed at acidic, neutral or alkaline pH and the oxidizing agent may be added to the composition of the invention just at the time of use, or it may be used starting with an oxidizing composition containing it, applied simultaneously or sequentially to the composition of the invention.

In one particular embodiment, the composition devoid of oxidizing agent according to the present invention is mixed, preferably at the time of use, with a composition containing, in a medium appropriate for dyeing, one or more oxidizing agents, these oxidizing agents being present in an amount sufficient to develop a colouring. The mixture obtained is then applied to the keratin fibres.

In accordance with this particular embodiment, a ready-to-use composition is obtained, which is a mixture of a composition according to the invention with one or more oxidizing agents.

After a leave-on time of from 3 to 50 minutes approximately and preferably 5 to 30 minutes approximately, the keratin fibres are rinsed, washed with shampoo, rinsed again and then dried.

The oxidizing agents are those indicated above.

The oxidizing composition may also contain various adjuvants conventionally used in compositions for dyeing the hair and as defined above.

The pH of the oxidizing composition containing the oxidizing agent is such that, after mixing with the dye composition, the pH of the resulting composition applied to the keratin fibres preferably varies between 3 and 12 approximately and more preferably still between 5 and 11. It may be adjusted to the desired value by means of acidifying or basifying agents usually used in the dyeing of keratin fibres and as defined previously.

The ready-to-use composition which is ultimately applied to the keratin fibres may be in a variety of forms, such as in the form of liquids, creams or gels or any other form appropriate for carrying out dyeing of keratin fibres, and in particular of human hair.

Another subject of the invention is a dyeing “kit” or multi-compartment device in which a first compartment contains the dye composition devoid of oxidizing agent of the present invention defined above, comprising one or more oxidation bases chosen from the compound of formula (I) or the addition salts thereof with an acid, and a second compartment contains one or more oxidizing agents.

These devices may be equipped with a means for dispensing the desired mixture on the hair, such as the devices described in patent FR-2 586 913 in the name of the Applicant.

The examples that follow serve to illustrate the invention without, however, being limiting in nature.

### EXAMPLES

### Example 1

### Synthesis of 2-(2,5-diaminophenoxy)-N,N,N-trimethylethanammonium chloride hydrochloride

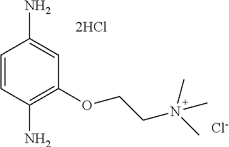

### Synthesis of 3-[2-(dimethylamino)ethoxy]-4-nitroaniline

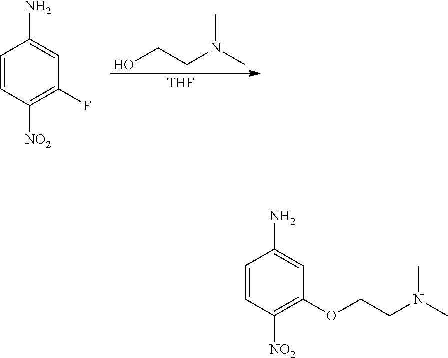

20 ml of THF are placed in a 50 mL three-necked flask equipped with a thermometer, a condenser and a bubbler, with magnetic stirring and under a stream of nitrogen. After cooling to 0° C., 2 mg (0.08 mol) of sodium hydride are added and this medium is stirred for 1 hour to obtain the alkoxide.

80 ml of THF are placed in a 100 ml three-necked flask equipped with a thermometer and a bubbler, with magnetic stirring and under a stream of nitrogen. After cooling to 0° C., 5.605 g (0.04 mol) of 3-fluoro-4-nitroaniline are added. The alkoxide solution prepared previously is added to this solution. The medium obtained is stirred for 24 hours while monitoring by TLC (9/1 CH₂Cl₂/MeOH).

The solvent is removed by evaporation on a rotavapor, followed by purification on a column of silica, eluting with dichloromethane/methanol.

The compound thus obtained in the form of a dark yellow powder, in a mass of 7.95 g (79% yield), corresponds to the expected compound.

Analysis by mass spectrometry confirms the expected structure:

The quasi-molecular ions [M+H]⁺ and [M+Na]⁺ of the expected molecule C₁₀H₁₅N₃O₃ are mainly detected.

### Synthesis of 2-(5-amino-2-nitrophenoxy)-N,N,N-trimethylethanaminium methyl sulfate

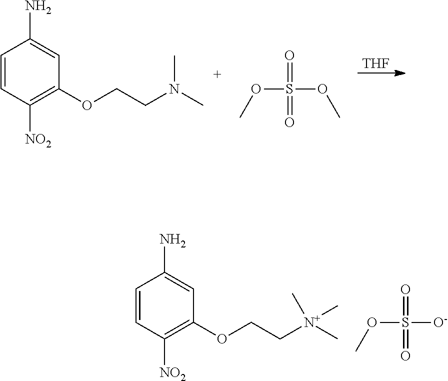

20 ml of THF and 1.67 g (0.007 mol) of 2-(2,5-diaminophenoxy)-N,N,N-trimethylethanaminium are successively placed in a 50 ml three-necked flask equipped with a thermometer, a condenser, a bubbler and a dropping funnel, with magnetic stirring. 0.739 ml (0.008 mol) of dimethyl sulfate is added to this solution and the medium, which is a clear solution, becomes heterogeneous, with precipitation of a yellow solid.

The medium is stirred overnight, and the solid formed is isolated by filtration and washing with ether.

The product is dried under vacuum in the presence of desiccant to constant weight. 2.13 g (87% yield) of the expected compound are thus obtained in the form of an orange powder.

Analysis by mass spectrometry confirms the expected compound and the expected cation [C₁₁H₁₈N₃O₃]⁺.

### Synthesis of 2-(2,5-diaminophenoxy)-N,N,N-trimethylethanaminium chloride dihydrochloride

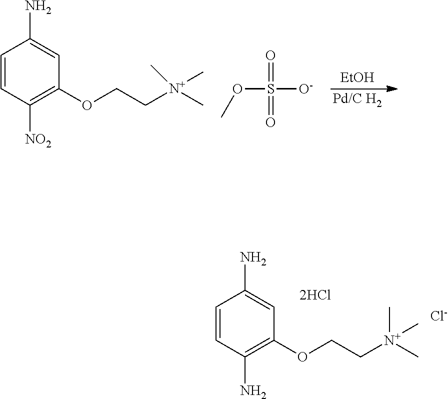

This reduction is carried out using an H-Cube hydrogenator containing a 90×4 mm cartridge of 10% Pd/C.

A solution derived from 3.95 g (0.017 mmol) of 2-(5-amino-2-nitrophenoxy)-N,N,N-trimethylethanaminium methyl sulfate in 300 ml of ethanol is introduced at a flow rate of 1 ml per minute onto a cartridge of palladium catalyst at 70° C. under a pressure of 65 bar in the H-Cube system in the presence of hydrogen.

At the machine outlet, the very pale yellow solution is added to a solution of 200 ml of 6N hydrochloric isopropanol. This solution is heated at 50° C. for 1 hour and the solvent is then removed by evaporation under vacuum.

The solid formed is recovered and is dried under vacuum at 30° C. in the presence of desiccant, to give 4.78 g of compound in the form of a grey powder.

The NMR (¹H 400 MHz and ¹³C 100.61 MHz DMSO-d₆) and mass spectrometry analyses are in accordance with the expected structure.

Elemental Analysis:

C(41.2); H(7.01); N(13.09); O(5.54); Cl(33.26)

### Example 2

### Synthesis of 1-[2-(2,5-diaminophenoxy)ethyl]-1-methylpiperidinium chloride dihydrochloride

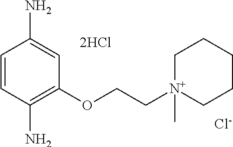 [SMILES: C[N+]1(CCOc2cc(N)ccc2N)CCCCC1 ; [Cl-]]

### Synthesis of 4-nitro-3-[2-(piperidin-1-yl)ethoxy]aniline

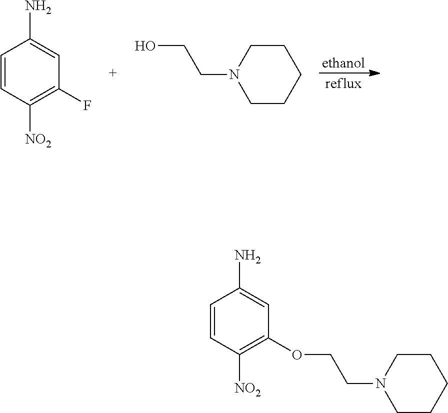

40 ml of THF are placed in a 50 ml three-necked flask equipped with a thermometer, a condenser and a bubbler, with magnetic stirring and under the stream of nitrogen. After cooling to 0° C., 3.436 mg (0.14 mol) of sodium hydride are added and this medium is stirred for 1 hour to obtain the alkoxide.

110 ml of THF are placed in a 100 ml three-necked flask equipped with a thermometer and a bubbler, with magnetic stirring and under a stream of nitrogen. After cooling to 0° C., 7.7 g (0.055 mol) of 3-fluoro-4-nitroaniline are added. The alkoxide solution prepared previously is added to this solution. The medium obtained is stirred for 24 hours while monitoring by TLC (9/1 CH₂Cl₂/MeOH).

The solvent is removed by evaporation on a rotavapor, followed by purification on a column of silica, eluting with dichloromethane/methanol.

The compound thus obtained in the form of a dark yellow powder, in a mass of 16.13 g (79% yield), corresponds to the expected compound.

Analysis by mass spectrometry confirms the structure of the expected compound. The quasi-molecular ions [M+H]⁺ and [M+Na]⁺ of the expected molecule C₁₃H₁₉N₃O₃ are mainly detected.

### Synthesis of 1-[2-(5-amino-2-nitrophenoxy)ethyl]-1-methylpiperidinium methyl sulfate

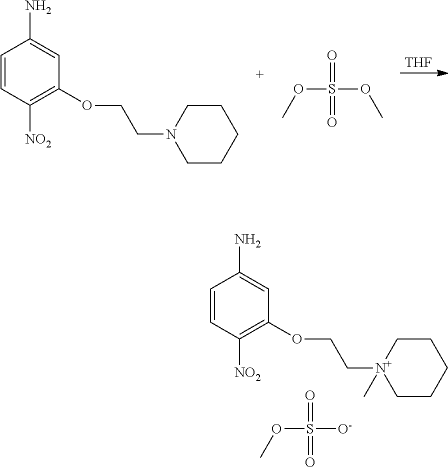

100 ml of THF and 8 g (0.07 mol) of 4-nitro-3-[2-(piperidin-1-yl)ethoxy]aniline are successively placed in a 50 ml three-necked flask equipped with a thermometer, a condenser, a bubbler and a dropping funnel, with magnetic stirring. 2.52 ml (0.032 mol) of dimethyl sulfate is added to this solution and the medium, which is a clear solution, becomes heterogeneous, with precipitation of a yellow solid.

The medium is stirred overnight, and the solid formed is isolated by filtration and washing with ether.

The product is dried under vacuum in the presence of desiccant to constant weight. 11.24 g (96% yield) of the expected compound are thus obtained in the form of a dark orange powder.

Analysis by mass spectrometry confirms the expected compound and the expected cation [C₁₄H₂₂N₃O₃]^{|}.

### Synthesis of 1-[2-(2,5-diaminophenoxy)ethyl]-1-methylpiperidinium chloride dihydrochloride

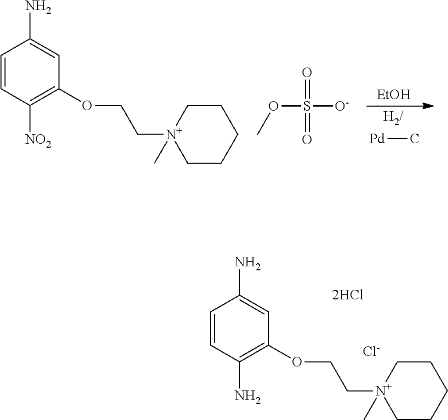

This reduction is carried out using an H-Cube hydrogenator containing a 90×4 mm cartridge of 10% Pd/C.

A solution derived from 11.24 g (0.028 mol) of 1-[2-(5-amino-2-nitrophenoxy)ethyl]-1-methylpiperidinium methyl sulfate in 1100 ml of ethanol is introduced at a flow rate of 15 ml per minute onto a cartridge of palladium catalyst at 70° C. under a pressure of 65 bar in the H-Cube system in the presence of hydrogen.

At the machine outlet, the very pale yellow solution is added to a solution of 200 ml of 6N hydrochloric isopropanol. This solution is maintained at 50° C. for 1 hour and the solvent is then removed by evaporation under vacuum.

The solid formed is recovered and is dried under vacuum at 30° C. in the presence of desiccant, to give 6.5 g (100% yield) of compound in the form of a light-beige powder.

The NMR (¹H 400 MHz and ¹³C 100.61 MHz DMSO-d₆) and mass spectrometry analyses are in accordance with the expected structure.

Elemental Analysis:

C(47.70); H(7.60); N(11.11); O(7.52); Cl(27.64)

### Example 3

### Synthesis of 4-[2-(2,5-diaminophenoxy)ethyl]-1,1-dimethylpiperazin-1-ium chloride dihydrochloride

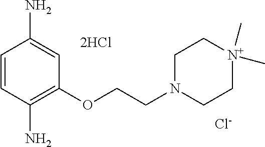 [SMILES: C[N+]1(C)CCN(CCOc2cc(N)ccc2N)CC1 ; [Cl-]]

### Synthesis of 2-[2-(4-methylpiperazin-1-yl)ethoxy]benzene-1,4-diamine

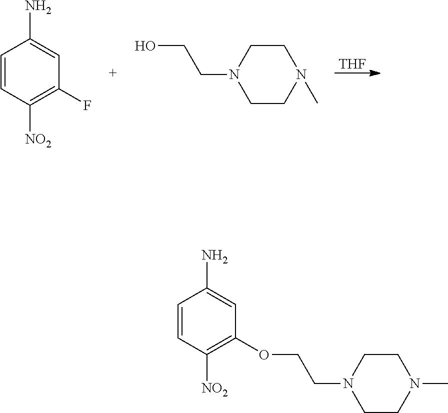

50 ml of THF and 5 g (0.153 mol) of 1-hydroxyethylpiperazine are placed in a 500 ml three-necked flask equipped with a thermometer, a condenser and a bubbler, with magnetic stirring and under a stream of nitrogen. After cooling to 0° C., 1.539 g (0.064 mol) of sodium hydride are added and this medium is stirred for 1 hour to obtain the alkoxide.

A solution derived from 250 ml of THF and 4.3 g (0.028 mol) of 3-fluoro-4-nitroaniline is added dropwise to this alkoxide. The medium obtained is stirred for 24 hours while monitoring by TLC (9/1 CH₂Cl₂/MeOH).

The solvent is removed by evaporation on a rotavapor, followed by purification on a column of silica, eluting with dichloromethane/methanol.

The compound thus obtained in the form of a yellow powder, in a mass of 9.3 g, corresponds to the expected compound.

Analysis by mass spectrometry confirms the structure of the expected compound. The quasi-molecular ions [M+H]⁺ and [M+Na]⁺ of the expected molecule C₁₃H₂₀N₄O₃ are mainly detected.

### Synthesis of 4-[2-(5-amino-2-nitrophenoxy)ethyl]-1,1-dimethylpiperazin-1-ium methyl sulfate

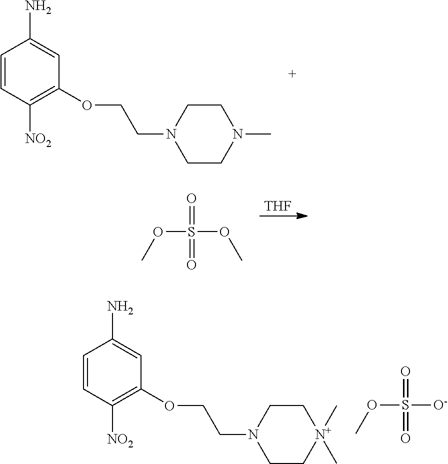

50 ml of THF and 8.8 g (0.031 mol) of 2-[2-(4-methylpiperazin-1-yl)ethoxy]benzene-1,4-diamine are successively placed in a 100 ml three-necked flask equipped with a thermometer, a condenser, a bubbler and a dropping funnel, with magnetic stirring. 3.126 ml (0.033 mol) of dimethyl sulfate are added to this solution and the medium, which is a clear solution, becomes heterogeneous, with precipitation of a yellow solid.

The medium is stirred overnight, and the solid formed is isolated by filtration and washing with ether.

The product is dried under vacuum in the presence of desiccant to constant weight. 4.31 g of the expected compound are thus obtained in the form of a dark orange powder.

Analysis by mass spectrometry confirms the expected compound, the expected cation [C₁₄H₁₃N₄O₃]⁺ is mainly detected.

### Synthesis of 4-[2-(2,5-diaminophenoxy)ethyl]-1,1-dimethylpiperazin-1-ium chloride dihydrochloride

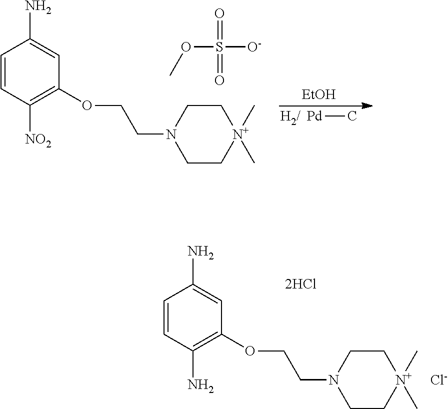

This reduction is carried out using an H-Cube hydrogenator containing a 90×4 mm cartridge of 10% Pd/C.

A solution derived from 9.9 g (0.024 mol) of 4-[2-(5-amino-2-nitrophenoxy)ethyl]-1,1-dimethylpiperazin-1-ium methyl sulfate in 1000 ml of ethanol is introduced at a flow rate of 15 ml per minute onto a cartridge of palladium catalyst at 70° C. under a pressure of 65 bar in the H-Cube system in the presence of hydrogen.

At the machine outlet, the very pale yellow solution is added to a solution of 200 ml of 6N hydrochloric isopropanol. This solution is heated at 50° C. for 1 hour and the solvent is then removed by evaporation under vacuum.

The solid formed is recovered and is dried under vacuum at 30° C. in the presence of desiccant, to give 6.2 g of compound in the form of a very pale egg-yellow powder.

The NMR (¹H 400 MHz and ¹³C 100.61 MHz DMSO-d₆) and mass spectrometry analyses are in accordance with the expected structure.

Elemental Analysis:

C(37.70); H(7.26); N(12.24); O(11.44); Cl(30.31)

### Examples 5

### Synthesis of 4-[2-(2,5-diaminophenoxy)ethyl]-1,1-dimethylpiperazin-1-ium chloride dihydrochloride

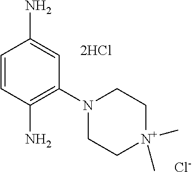 [SMILES: C[N+]1(C)CCN(c2cc(N)ccc2N)CC1 ; [Cl-]]

### Synthesis of 3-(4-methylpiperazin-1-yl)-4-nitroaniline

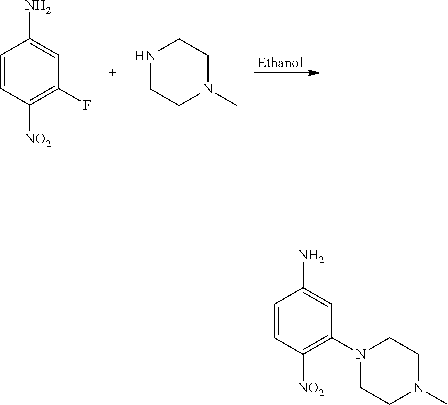

50 ml of ethanol and 6.4 g (0.064 mol) of 1-methylpiperazine are placed in a 100 ml three-necked flask equipped with a thermometer, a condenser and a bubbler, with magnetic stirring and under a stream of nitrogen. The medium is refluxed for 2 hours.

The medium is poured onto a mixture of ice and water, and a yellow solid precipitates. After stirring for 30 minutes, the yellow solid is drained by suction, washed with water and dried under vacuum at 30° C. in the presence of a desiccant to constant weight.

The compound thus obtained in the form of a yellow powder, in a mass of 6.5 g, corresponds to the expected compound.

Analysis by mass spectrometry confirms the structure of the expected compound. The quasi-molecular ions [M+H]⁺ and [M+Na]⁺ of the expected molecule C₁₁H₁₆N₄O₂ are mainly detected.

### Synthesis of 4-(5-amino-2-nitrophenyl)-1,1-dimethylpiperazin-1-ium methyl sulfate

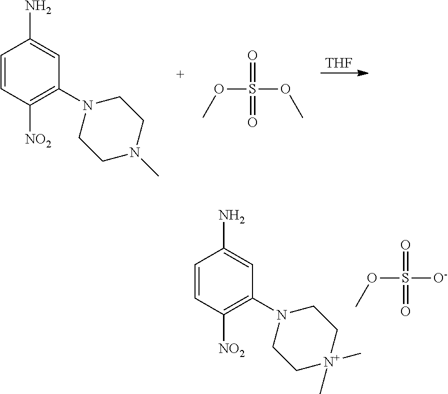

100 ml of THF and 5.188 g (0.022 mol) of 3-(4-methylpiperazin-1-yl)-4-nitroaniline are successively placed in a 250 ml three-necked flask equipped with a thermometer, a condenser, a bubbler and a dropping funnel, with magnetic stirring. 2.187 ml (0.023 mol) of dimethyl sulfate are added to this solution and the medium, which is a clear solution, becomes heterogeneous, with precipitation of a yellow solid.

The medium is stirred overnight, and the solid formed is isolated by filtration and washing with ether.

The product is dried under vacuum in the presence of desiccant to constant weight. 7.67 g of the expected compound are thus obtained in the form of a dark orange powder.

Analysis by mass spectrometry confirms the expected compound, the expected cation [C₁₂H₁₉N₄O₂]⁺ is mainly detected.

### Synthesis of 4-(2,5-diaminophenyl)-1,1-dimethylpiperazin-1-ium chloride dihydrochloride

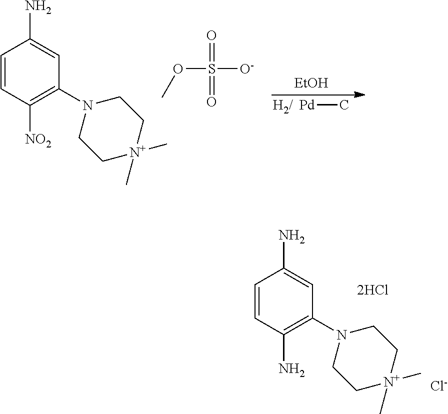

This reduction is carried out using an H-Cube hydrogenator containing a 90×4 mm cartridge of 10% Pd/C.

A solution derived from 5.2 g (0.022 mol) of 4-(5-amino-2-nitrophenyl)-1,1-dimethylpiperazin-1-ium methyl sulfate in 1000 ml of ethanol is introduced at a flow rate of 15 ml per minute onto a cartridge of palladium catalyst at 70° C. under a pressure of 65 bar in the H-Cube system in the presence of hydrogen.

At the machine outlet, the very pale yellow solution is added to a solution of 200 ml of 6N hydrochloric isopropanol. This solution is heated at 50° C. for 1 hour and the solvent is then removed by evaporation under vacuum.

The solid formed is recovered and is dried under vacuum at 30° C. in the presence of desiccant, to give 6.2 g (85% yield) of compound in the form of a grey powder.

The NMR (¹H 400 MHz and ¹³C 100.61 MHz DMSO-d₆) and mass spectrometry analyses are in accordance with the expected structure.

Elemental Analysis:

C(43.3); H(7.07); N(16.66); Cl(31.14)

Examples of Dyeing

The following dye compositions (A) to (P) are prepared from the ingredients below:

| Compositions | A | B | C | D |
|---|---|---|---|---|
| 2-(2,5-diaminophenoxy)- | 10⁻³ mol | 10⁻³ mol | 10⁻³ mol | 10⁻³ mol |
| N,N,N-trimethylethanaminium | | | | |
| chloride dihydrochloride | | | | |
| 6-hydroxybenzomorpholine | 10⁻³ mol | | | |
| 2-methyl-5- | | 10⁻³ mol | | |
| hydroxyethylaminophenol | | | | |
| 2-amino-3-hydroxypyridine | | | 10⁻³ mol | |
| 3-amino-2-chloro-6- | | | | 10⁻³ mol |
| methylphenol | | | | |
| Dye support (1) | (*) | (*) | (*) | (*) |
| Demineralized water qs | 100 g | 100 g | 100 g | 100 g |
| Shade observed | green | violet | brown | strong |
| | Strong | strong | | violet- |
| | chromatic | chromatic | | blue |

| Compositions | E | F | G | H |
|---|---|---|---|---|
| 1-[2-(2,5- | 10⁻³ mol | 10⁻³ mol | 10⁻³ mol | 10⁻³ mol |
| diaminophenoxy)ethyl]-1- | | | | |
| methylpiperidinium chloride | | | | |
| dihydrochloride | | | | |
| 6-hydroxybenzomorpholine | 10⁻³ mol | | | |
| 2-methyl-5- | | 10⁻³ mol | | |
| hydroxyethylaminophenol | | | | |
| 2-amino-3-hydroxypyridine | | | 10⁻³ mol | |
| 3-amino-2-chloro-6- | | | | 10⁻³ mol |
| methylphenol | | | | |
| Dye support (1) | (*) | (*) | (*) | (*) |
| Demineralized water qs | 100 g | 100 g | 100 g | 100 g |
| Shade observed | yellow- | violet | light- | strong |
| | green | strong | brown | violet- |
| | chromatic | | | blue |
| | strong | | | |
| Compositions | I | J | K | L |
| 4-(3,5-diaminopyridin-2-yl)- | 10⁻³ mol | 10⁻³ mol | 10⁻³ mol | 10⁻³ mol |
| 1,1-dimethylpiperazin-1-ium | | | | |
| chloride dihydrochloride | | | | |
| 6-hydroxybenzomorpholine | 10⁻³ mol | | | |
| 2-methyl-5- | | 10⁻³ mol | | |
| hydroxyethylaminophenol | | | | |
| 2-amino-3-hydroxypyridine | | | 10⁻³ mol | |
| 3-amino-2-chloro-6- | | | | 10⁻³ mol |
| methylphenol | | | | |
| Dye support (1) | (*) | (*) | (*) | (*) |
| Demineralized water qs | 100 g | 100 g | 100 g | 100 g |
| Shade observed | strong | pale | strong | chromatic |
| | green- | violet | brown | violet |
| | yellow | | | strong |

| Compositions | M | N | O | P |
|---|---|---|---|---|
| 4-[2-(2,5- | 10⁻³ mol | 10⁻³ mol | 10⁻³ mol | 10⁻³ mol |
| diaminophenoxy)ethyl]-1,1- | | | | |
| dimethylpiperazin-1- | | | | |
| ium chloride | | | | |
| dihydrochloride | | | | |
| 6- | 10⁻³ mol | | | |
| hydroxybenzomorpholine | | | | |
| 2-methyl-5- | | 10⁻³ mol | | |
| hydroxyethylaminophenol | | | | |
| 2-amino-3- | | | 10⁻³ mol | |
| hydroxypyridine | | | | |
| 3-amino-2-chloro-6- | | | | 10⁻³ mol |
| methylphenol | | | | |
| Dye support (1) | (*) | (*) | (*) | (*) |
| Demineralized water qs | 100 g | 100 g | 100 g | 100 g |
| Shade observed | pale green- | strong | strong | chromatic |
| | yellow | violet | brown | blue- |
| | | | | violet |
| | | | | strong |

### pH 9.5 Dye Support (1)

| | |
|---|---|
| 96° ethyl alcohol | 20.8 g |
| 35% aqueous sodium metabisulfite solution | 0.23 g AM |
| pentasodium salt of | 0.48 g AM |
| diethylenetriaminepentaacetic acid as an | |
| aqueous 40% solution | |
| C₈-C₁₀ alkyl polyglucoside as an | 3.6 g AM |
| aqueous 60% solution | |
| benzyl alcohol | 2.0 g |
| polyethylene glycol containing 8 units of ethylene | 3.0 g |
| oxide | |
| NH₄Cl | 4.32 g |
| aqueous ammonia containing 20% NH₃ | 2.94 g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After leaving the mixture on for 30 minutes, the locks are rinsed, washed with a standard shampoo, rinsed again and then dried to give the various shades.

1-13. (canceled)

14. A cationic para-phenylenediamine of formula (I), the optical isomers and geometrical isomers thereof, the addition salts thereof and/or the solvates thereof: 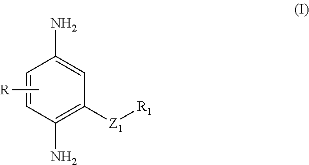
wherein:
R is chosen from a hydrogen atom, a halogen atom, a C₁-C₄ alkyl radical, a carboxyl radical, and a (C₁-C₄)alkoxycarbonyl radical;
Z₁ is chosen from an oxygen atom and a group NR₆;
R₆ is chosen from a hydrogen atom and a linear or branched C₁-C₄ alkyl radical;
R₁ is chosen from a linear or branched C₁-C₁₀ alkyl radical, which is substituted with a cationic radical; or a saturated, unsaturated or aromatic 5- to 8-membered non-cationic ring or heterocycle substituted with a cationic radical;
when Z₁ represents NR₆ then R₁ and R₆ may form, together with the nitrogen atom to which they are attached, a saturated or unsaturated 5- to 8-membered cationic heterocycle; or a saturated or unsaturated, 5- to 8-membered noncationic heterocycle; and
the cationic radical is a linear or branched or non aromatic heterocyclic radical comprising a quaternary ammonium, the quaternary ammonium being of the type —N⁺RₐR_{b}R_{c}, wherein:
Rₐ, R_{b} and R_{c}, which may be identical or different, are chosen from a C₁-C₆ alkyl radical, or
Rₐ and R_{b} can together form a heterocycle comprising 5 to 8 ring members, and the radical R_{c} is chosen from a C₁-C₆ alkyl radical.

15. The cationic para-phenylenediamine of formula (I) according to claim 14, wherein:
R is chosen from a hydrogen atom and a C₁-C₄ alkyl radical,
Z₁ is chosen from an oxygen atom and a group NR₆ with R₆ representing a hydrogen atom or a C₁-C₂ alkyl radical,
R₁ is chosen from a linear or branched C₁-C₈ alkyl radical, substituted with a cationic radical; and a saturated, unsaturated or aromatic, 5- to 8-membered non-cationic ring substituted with a cationic radical, and
when Z₁ represents NR₆ then R₁ and R₆ may form, together with the nitrogen atom to which they are attached, a saturated or unsaturated cationic 5- to 8-membered heterocycle, it being possible for the heterocycle to contain one or more heteroatoms chosen from N or O; or a saturated or unsaturated non-cationic 5- to 8-membered heterocycle substituted with a cationic radical.

16. The cationic para-phenylenediamine of formula (I) according to claim 14, wherein the cationic radical is chosen from trimethylammonium, trimethylammonium and tri-β-hydroxyethylammonium radicals.

17. The cationic para-phenylenediamine of formula (I) according to claim 14, wherein the cationic heterocycle is chosen from piperazinium, pyrrolidinium, morpholinium, pyrrolidine, piperidine, morpholine and piperazine rings.

18. The cationic para-phenylenediamine of formula (I) according to claim 14, wherein:
R is a hydrogen atom,
Z₁ is chosen from an oxygen atom and a group NH or NCH₃, and
R₁ is a saturated linear C₂-C₈ alkyl radical substituted with a cationic radical chosen from trimethylammonium radicals and piperazinium, pyrrolidinium, piperidinium or morpholinium cationic heterocycles.

19. The cationic para-phenylenediamine of formula (I) according to claim 14, wherein:
R is a hydrogen atom,
Z₁ is a group NR₆, and
R₁ and R₆ form, together with the nitrogen atom to which they are attached, a piperazinium, pyrrolidinium, piperidinium or morpholinium radical substituted with at least one radical chosen from C₁-C₄ hydroxyalkyl and C₁-C₄ alkyl radicals.

20. The cationic para-phenylenediamine of formula (I) according to claim 14, wherein:
R is a hydrogen atom,
Z₁ is a group NR₆, and
R₁ and R₆ form, together with the nitrogen atom to which they are attached, a saturated or unsaturated, 5- to 8-membered non-cationic heterocycle substituted with a cationic radical.

21. The cationic para-phenylenediamine of formula (I) according to claim 14, wherein the compound is chosen from the following compounds and the geometrical or optical isomer forms thereof, the tautomers thereof, the organic or mineral acid or base salts thereof, and the solvates thereof:

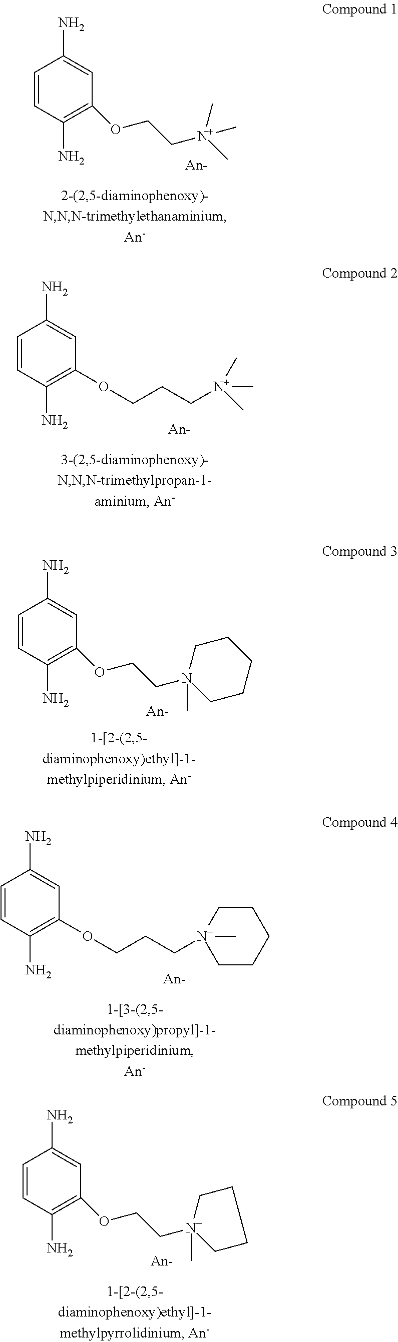

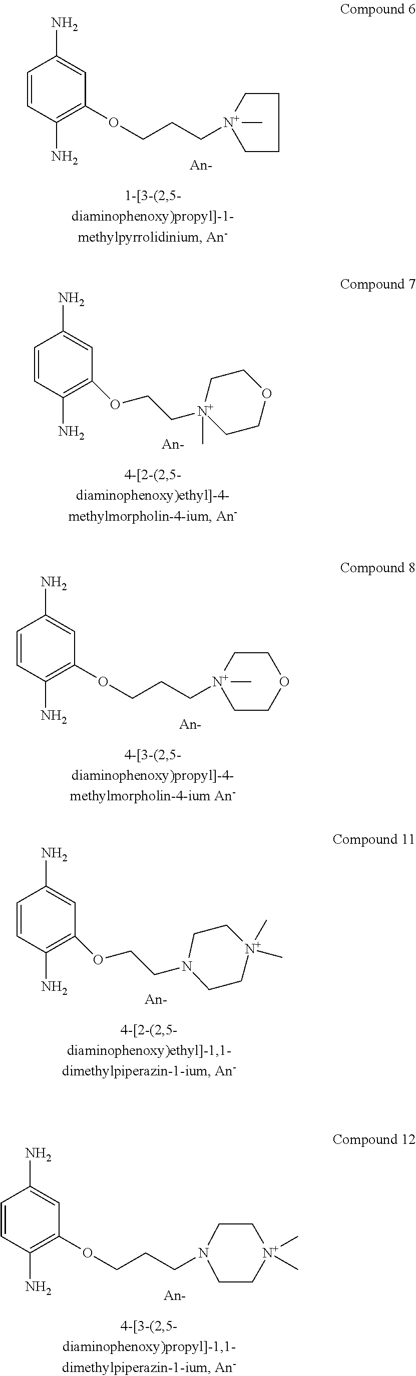

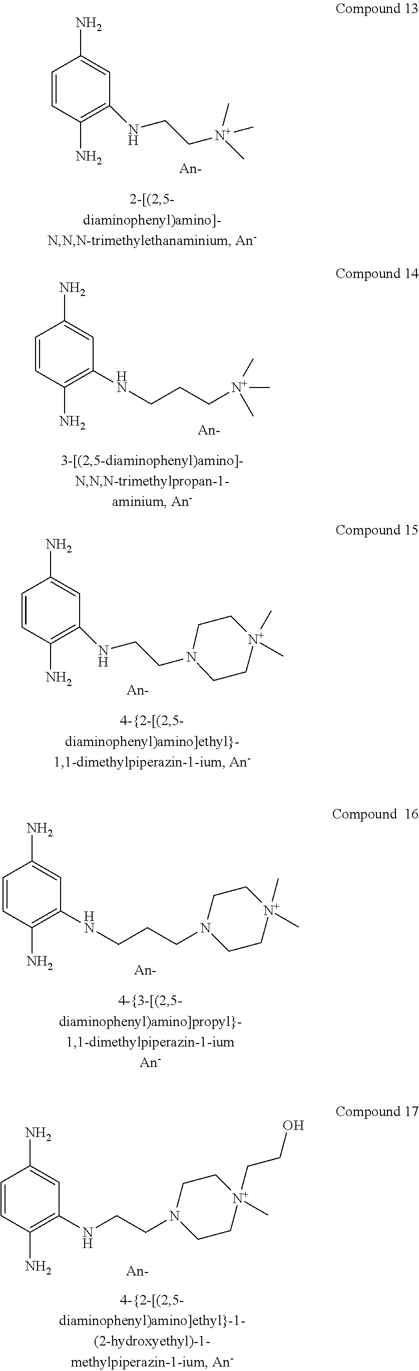 [SMILES: C[N+](C)(C)CCNc1cc(N)ccc1N ; C[N+](C)(C)CCCNc1cc(N)ccc1N ; C[N+]1(C)CCN(CCNc2cc(N)ccc2N)CC1 ; C[N+]1(C)CCN(CCCNc2cc(N)ccc2N)CC1 ; C[N+]1(CCO)CCN(CCNc2cc(N)ccc2N)CC1]

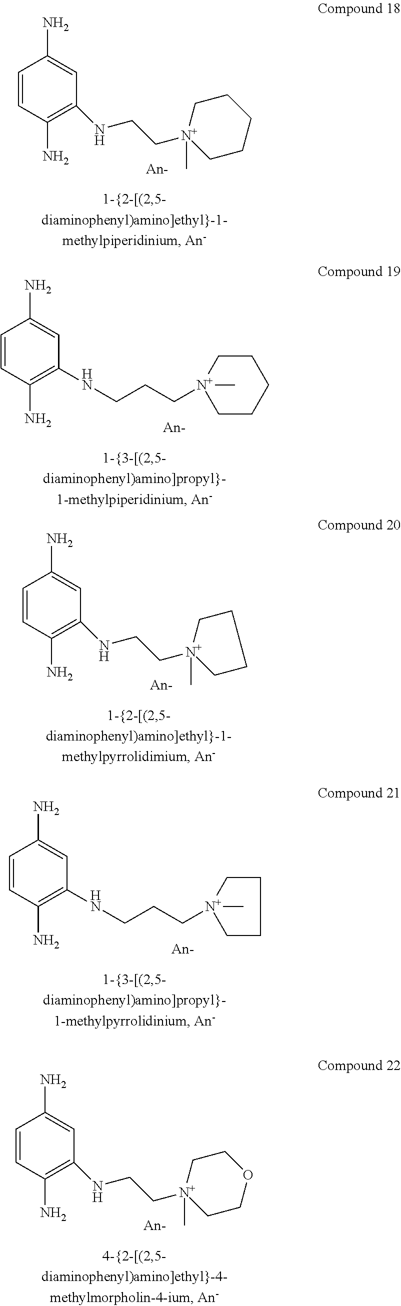

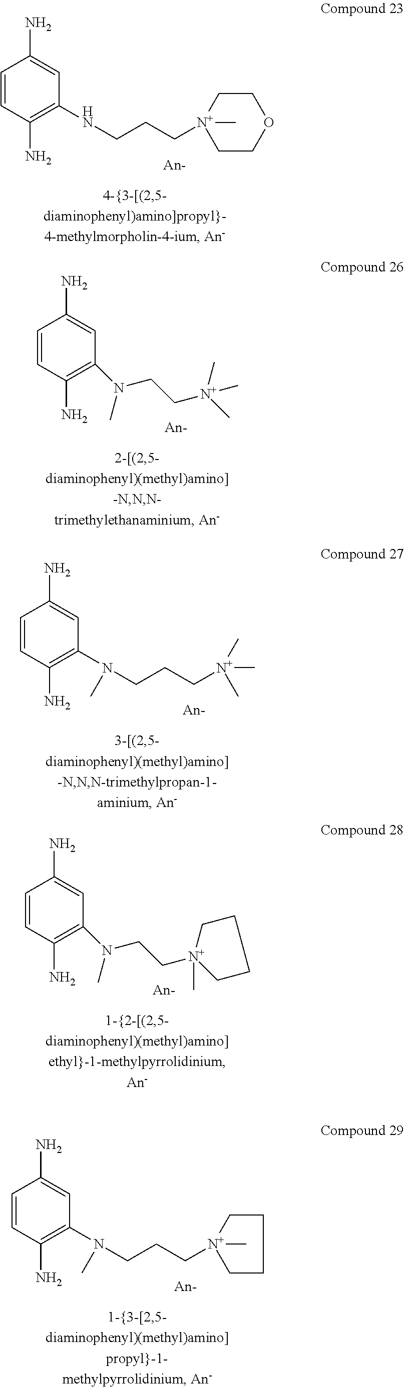

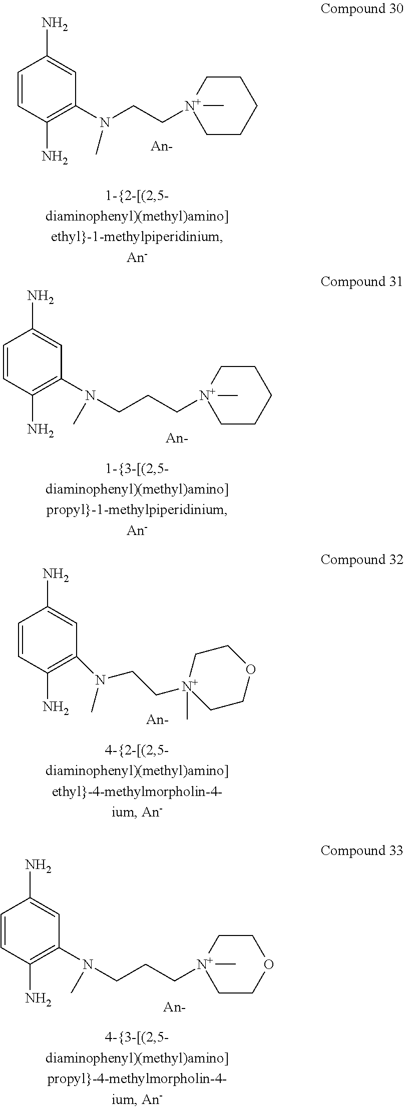

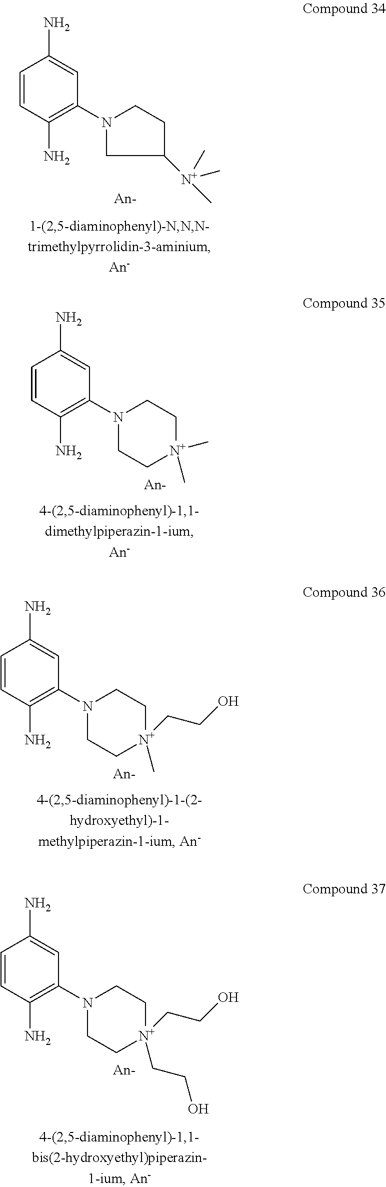 [SMILES: C[N+](C)(C)C1CCN(c2cc(N)ccc2N)C1 ; C[N+]1(C)CCN(c2cc(N)ccc2N)CC1 ; C[N+]1(CCO)CCN(c2cc(N)ccc2N)CC1 ; Nc1ccc(N)c(N2CC[N+](CCO)(CCO)CC2)c1]

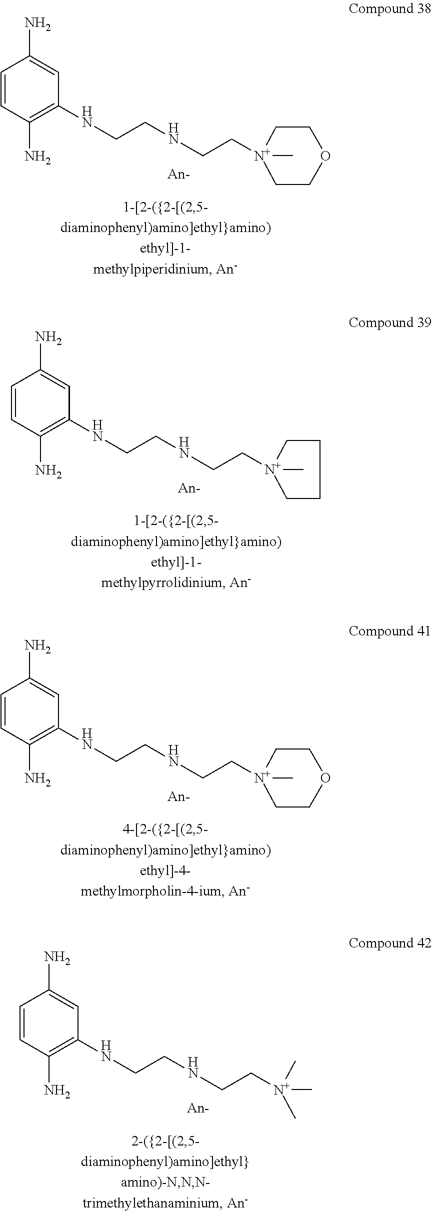

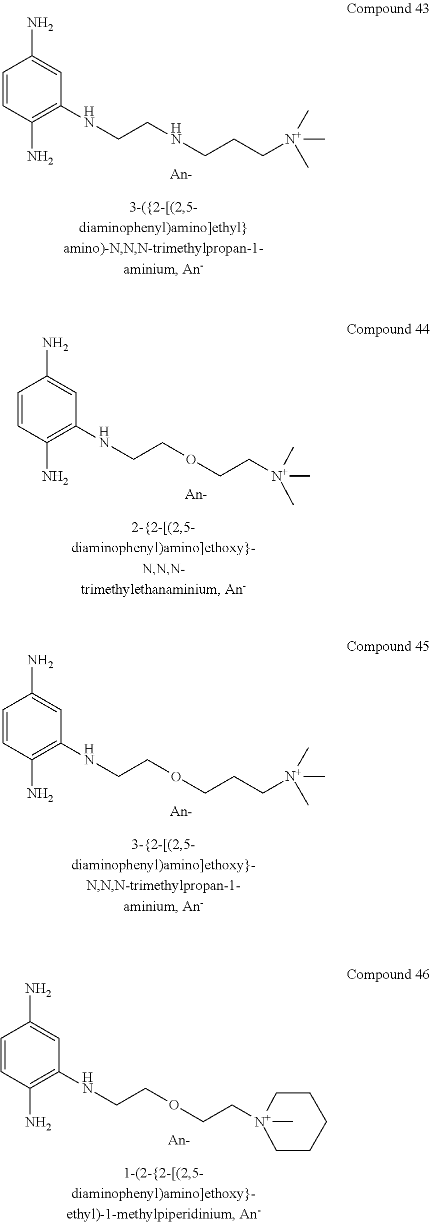

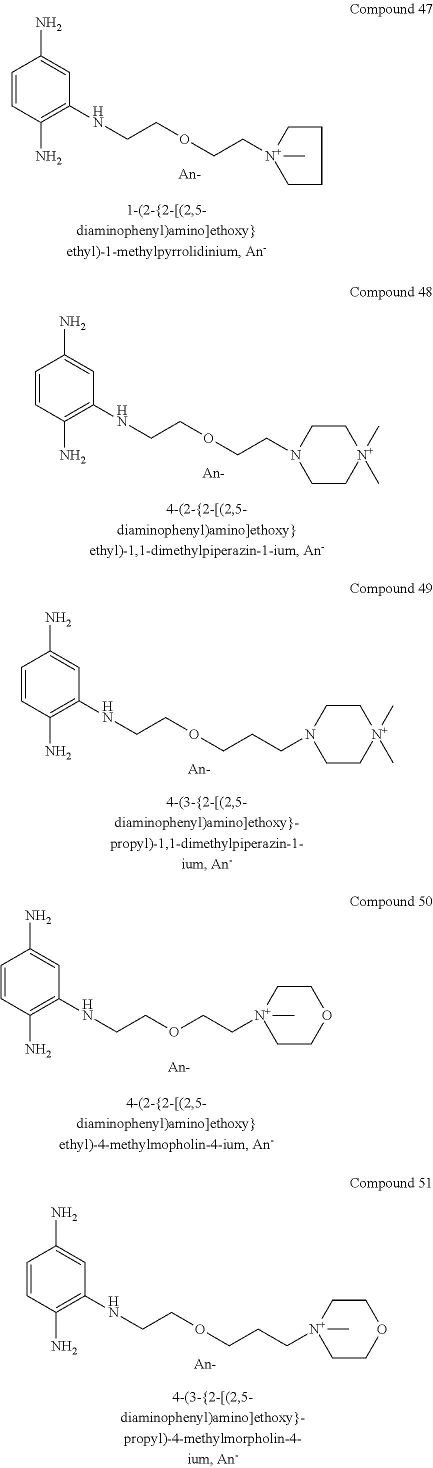

22. A composition for dyeing keratin fibers, comprising, in a suitable dyeing medium, at least one cationic para-phenylenediamine of formula (I): 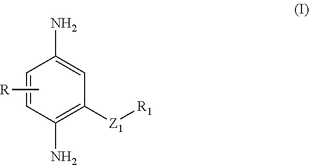
wherein:
R is chosen from a hydrogen atom, a halogen atom, a C₁-C₄ alkyl radical, a carboxyl radical, and a (C₁-C₄)alkoxycarbonyl radical;
Z₁ is chosen from an oxygen atom and a group NR₆;
R₆ is chosen from a hydrogen atom and a linear or branched C₁-C₄ alkyl radical;
R₁ is chosen from a linear or branched C₁-C₁₀ alkyl radical, which is substituted with a cationic radical; or a saturated, unsaturated or aromatic 5- to 8-membered non-cationic ring or heterocycle substituted with a cationic radical;
when Z₁ represents NR₆ then R₁ and R₆ may form, together with the nitrogen atom to which they are attached, a saturated or unsaturated 5- to 8-membered cationic heterocycle; or a saturated or unsaturated, 5- to 8-membered noncationic heterocycle; and
the cationic radical is a linear or branched or non aromatic heterocyclic radical comprising a quaternary ammonium, the quaternary ammonium being of the type —N⁺RₐR_{b}R_{c}, wherein:
Rₐ, R_{b} and R_{c}, which may be identical or different, are chosen from a C₁-C₆ alkyl radical, or
Rₐ and R_{b} can together form a heterocycle comprising 5 to 8 ring members, and the radical R_{c} is chosen from a C₁-C₆ alkyl radical.

23. The composition according to claim 22 further comprising at least one coupler and/or at least one oxidation base other than the para-phenylenediamine of claim 22.

24. A method for dyeing keratin fibers, the method comprising:
applying to the keratin fibers in the presence of at least one oxidizing agent for a time that is sufficient to obtain the desired coloration, a dye composition comprising a cationic para-phenylenediamine of formula (I): 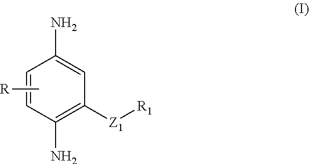
wherein:
R is chosen from a hydrogen atom, a halogen atom, a C₁-C₄ alkyl radical, a carboxyl radical, and a (C₁-C₄)alkoxycarbonyl radical;
Z₁ is chosen from an oxygen atom and a group NR₆;
R₆ is chosen from a hydrogen atom and a linear or branched C₁-C₄ alkyl radical;
R₁ is chosen from a linear or branched C₁-C₁₀ alkyl radical, which is substituted with a cationic radical; or a saturated, unsaturated or aromatic 5- to 8-membered non-cationic ring or heterocycle substituted with a cationic radical;
when Z₁ represents NR₆ then R₁ and R₆ may form, together with the nitrogen atom to which they are attached, a saturated or unsaturated 5- to 8-membered cationic heterocycle; or a saturated or unsaturated, 5- to 8-membered noncationic heterocycle; and
the cationic radical is a linear or branched or non aromatic heterocyclic radical comprising a quaternary ammonium, the quaternary ammonium being of the type —N⁺RₐR_{b}R_{c}, wherein:
Rₐ, R_{b} and R_{c}, which may be identical or different, are chosen from a C₁-C₆ alkyl radical, or
Rₐ and R_{b} can together form a heterocycle comprising 5 to 8 ring members, and the radical R_{c} is chosen from a C₁-C₆ alkyl radical;
optionally rinsing the keratin fibers;
optionally washing the keratin fibers with shampoo;
optionally rinsing the keratin fibers; and
drying the keratin fibers or leaving the keratin fibers to dry.

25. A multi-compartment device comprising a first compartment configured to contain the dye composition according to claim 22 and a second compartment configured to contain at least one oxidizing agent.

26. A multi-compartment dyeing kit comprising a first compartment configured to contain the dye composition according to claim 22 and a second compartment configured to contain at least one oxidizing agent.
